(19) United States
(12) Patent Application Publication
(10) Pub. No.: US 2024/0188859 A1
(43) Pub. Date: Jun. 13, 2024

(54) Monitoring Medical System with UV Exposure

(71) Applicant: Bard Access Systems, Inc., Salt Lake City, UT (US)

(72) Inventors: Stefan Josef Fellner, Salt Lake City, UT (US); Dustin Payne, West Jordan, UT (US); Steffan Sowards, Salt Lake City, UT (US); William Robert McLaughlin, Bountiful, UT (US)

(21) Appl. No.: 18/080,624

(22) Filed: Dec. 13, 2022

(51) Int. Cl. A61B 5/1459; A61N 5/06

(52) U.S. Cl. CPC A61B 5/1459; A61N 5/0601; A61N 2005/0661

(57) ABSTRACT

A system for treating infections in patients that includes an elongate probe configured for insertion into the patient body and a system module coupled with the probe. Sensors are disposed along the probe that detect infectious substances or physiological symptoms of the patient. The probe also includes light disseminating devices configured to expose the infectious substances to a light capable of disrupting the infectious substances, such as light within the ultraviolet spectrum. System logic may activate the light disseminating devices in response to sensor signals. A wireless connection enables clinician interaction with the system via external computing devices. The sensors may be disposed on external surfaces or internal luminal surfaces of the probe. The light disseminating devices may project the light away from the external surfaces or within a lumen of the probe. The light disseminating devices may include optical fibers.

### BACKGROUND

Modern healthcare employs many types of invasive devices and procedures to treat patients and to help them recover. Infections can be associated with the devices used in medical procedures, such as catheters. These healthcare-associated infections include central line-associated bloodstream infections and catheter-associated urinary tract infections. In some severe cases, bacterial infection may cause sepsis. In some cases, infections may be present within the blood. In short, patient infections present a significant risk to patients and may result in extended hospital stays and greater healthcare costs. Furthermore, infections may cause biofilms to build up on medical device surfaces which may affect operation of the medical device. While antibiotics may treat bacterial infections generally, biofilms are resistant to antibiotics.

Disclosed herein are systems and methods that address the foregoing.

### SUMMARY

Disclosed herein is a medical system that, according to some embodiments, includes an elongate probe configured for insertion into a patient body, where the elongate probe includes a number of light disseminating devices. The system further includes a number of sensors configured to operatively couple with the patient body and a system module coupled with the elongate probe. The system module includes a console operatively coupled with the sensors and the light disseminating devices, where the console includes a processor and a memory having stored thereon logic that, when executed by the processor, causes operations of the system. The operations include receiving a sensor signal from the sensors and activating the light disseminating devices in response to a triggering event, where a light disseminated by the light disseminating devices is configured to denature an infectious substance adjacent the elongate probe.

In some embodiments, the light is composed of wavelengths within at least one of a blue, violet, or ultraviolet spectrum.

In some embodiments, the console is communicatively coupled with an external computing device, and the operations further include (i) receiving input from the external computing device and (ii) transmitting system operational information to the external computing device.

In some embodiments, the system module is configured to selectively attach to and detach from the elongate probe, where attachment of the system module to the elongate probe includes operatively coupling the console with the sensors and the light disseminating devices.

In some embodiments, at least a subset of the sensors are coupled with an outside surface of the elongate probe. In some embodiments, the light disseminating devices are configured to project a light radially away from the elongate probe.

In some embodiments, the elongate probe includes a number of optical fibers extending along the elongate probe, where the optical fibers including the light disseminating devices. In such embodiments, the console includes a light source coupled with the optical fibers, and activating the light disseminating devices includes activating the light source.

In some embodiments, the elongate probe includes a stylet configured for placement within a lumen of a catheter. In some embodiments, the elongate probe includes a catheter, where the optical fibers are embedded within a luminal wall of the catheter and in some embodiments, at least a subset of the sensors are coupled to an inside luminal wall surface of the catheter. In some embodiments, the light disseminating devices are configured to project the light radially inward from the luminal wall. In some embodiments, activating the light disseminating devices is configured to denature a biofilm disposed within the lumen.

In some embodiments, the triggering event includes a physical manipulation of the system module by a clinician and in some embodiments, the triggering event includes an input received from the external computing device.

In some embodiments, the operations further include comparing a magnitude of a physiological parameter of the patient based on the sensor signal with a limit of the physiological parameter stored in the memory, and the triggering event includes a result of the comparison indicating that the magnitude exceeds the limit.

In some embodiments, the physiological parameter includes at least one of a redness skin color, a swelling, a temperature, a pH, a white blood cell count, a blood pressure or a lactate level. In some embodiments, at least a subset of the sensors are coupled to a skin surface of the patient.

In some embodiments, the sensor signal includes a detection level of the substance adjacent the elongate probe, and the operations further include comparing the detection level with a detection level limit stored in the memory. In such embodiments, the triggering event includes a result of the comparison indicating that the detection level exceeds the detection level limit.

Also disclosed herein is a method of treating a patient infection that, according to some embodiments, includes (i) inserting an elongate probe within vasculature of the patient, where the elongate probe includes a number of sensors. The method further includes, detecting via the number of sensors, at least one of (i) a physiological parameter of the patient exceeding a predefined limit or (ii) a detection level of an infectious substance exceeding a predefined detection level limit. The method further includes activating a number light disseminating devices coupled with the elongate probe, where a light disseminated by the light disseminating devices is configured to denature the infectious substance adjacent the elongate probe.

Also disclosed herein is a method of maintaining patency of a catheter that, according to some embodiments, includes (i) detecting, via a number of sensors coupled with an inside surface of a luminal wall the catheter, a biofilm disposed along the inside surface and (ii) activating a number light disseminating devices coupled with the catheter to project a light away from the inside surface onto the biofilm, the light configured to denature the biofilm.

These and other features of the concepts provided herein will become more apparent to those of skill in the art in view of the accompanying drawings and following description, which describe particular embodiments of such concepts in greater detail.

### DRAWINGS

FIG. 1 illustrates a medical system including an elongate probe, in accordance with some embodiments.

FIG. 2 is a block diagram of a console of the system of FIG. 1, in accordance with some embodiments.

FIG. 3A illustrates the system of FIG. 1 in use with a patient, in accordance with some embodiments.

FIG. 3B illustrates a detailed view of a distal portion of the elongate probe disposed within a blood vessel of the patient, in accordance with some embodiments.

FIG. 4 illustrates a flow chart of an exemplary method for utilizing the system of FIG. 1 to treat a patient infection, in accordance with some embodiments.

FIG. 5A illustrates another embodiment of the system having an elongate probe in the form of a catheter, in accordance with some embodiments.

FIG. 5B is detailed cross-sectional side view of a portion of the catheter of the FIG. 5A, in accordance with some embodiments.

FIG. 5C is detailed cross-sectional end view of the catheter of the FIG. 5A, in accordance with some embodiments.

FIG. 6 illustrates a flow chart of an exemplary method for disrupting a biofilm disposed on a luminal surface of the catheter of FIGS. 5A-5C, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The terms “proximal” and “distal” refer to opposite ends of a medical device, including the devices disclosed herein. As used herein, the proximal portion of an elongate probe is the portion nearest a practitioner during use, while the distal portion is the portion at the opposite end. For example, the distal end of the elongate probe is defined as the end closest to the patient during utilization of the elongate probe. The proximal end is the end opposite the distal end.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

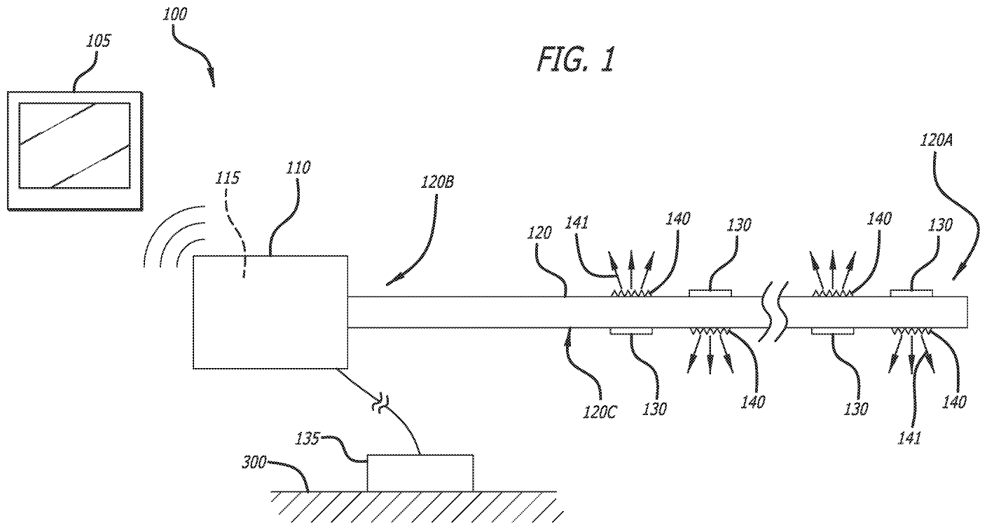

FIG. 1 illustrates a medical system (system) 100, according to some embodiments. The system 100 is generally configured to provide or enhance a treatment for a patient, such as treating a patient infection, for example. More specifically, the system 100 is configured to inhibit or reduce infection or the symptoms of infection, such as sepsis, for example. The system 100 may detect the presence or level of the infection and project a light onto infection substances to denature, disrupt, or otherwise reduce the effects of the infection.

The system 100 includes a system module 110 coupled with an elongate probe 120 defining a distal end 120A and a proximal end 120B. The elongate probe 120 is configured for insertion into a patient body where the desired effects of the light may be applied. The system module 110 is configured for use outside the patient body and may be coupled with the elongate probe 120 adjacent the proximal end 120B. The system module 110 includes a console 115 which in some embodiments, may be configured to wirelessly exchange data and/or information with an external computing device 105, such as a network computer, a tablet, or a cell phone, for example. In some embodiments, the computing device 105 may be coupled with or otherwise associated with an electronic medical records system.

The system module 110 may be configured to selectively attach to and detach from the elongate probe 120. In some embodiments, the elongate probe 120 may be configured for single use and the system module 110 for multiple uses. For example, the system module 110 may be configured to individually couple with a number (e.g., 2, 5, 10, 20 or more) of elongate probes 120.

The elongate probe 120 may be coupled to or otherwise associated with a ureteroscope, endoscope, a guidewire, a stylet or any other suitable device configured for insertion into the patient body. In some embodiments, the elongate probe 120 may be configured for placement within a lumen of a catheter, such as a vascular catheter or a urinary catheter, for example. The system 100 may be especially helpful when employed with a central catheter since the central catheter may be inserted into the patient body for an extend period of time, e.g., several days. In some embodiments, the system 100 may be configured to extend the useful life of the central catheter. For example, the system 100 may be configured to maintain a patency of the central catheter.

The system 100 includes a number (e.g., 1, 2, 3, 4 or more) of sensors 130 configured to detect or measure parameters associated with the patient. The sensors 130 are communicatively coupled with the console 115 so that the console 115 may receive and process electrical signals from the sensors 130. In some embodiments, at least a subset of the sensors 130 may be disposed along the elongate probe 120. In some embodiments, at least a subset of the sensors 130 may be located on an exterior surface 120C of the elongate probe 120. The sensors 130 may take on any suitable form factor. For example, a sensor 130 may take the form of a patch disposed on a side of the elongate probe 120 or a sensor 130 may take form of a band extending around the elongate probe 120.

The sensors 130 are configured to operatively couple with the patient via any suitable mechanism, i.e., the sensors 130 may fluidly, optically, electrically, thermally, physically, or biologically couple with the patient. In some embodiments, a subset 135 of the sensors 130 may be physically coupled with an external skin surface of the patient.

In some embodiments, at least a subset of the sensors 130 are configured to detect or measure one or more physiological parameters of the patient. In some embodiments, the physiological parameters may include a change in color of bodily tissue, such as a redness of the skin for example. The physiological parameters may include a change in shape of bodily tissue, such as a swelling, for example. Additional physiological parameters may include a temperature, a pH, a white blood cell count, a blood pressure or a lactate level of the patient. In some embodiments, the physiological parameters may be symptomatically associated with an infection of the patient.

In some embodiments, at least a subset of the sensors 130 are configured to detect the presence or level of a substance associated with an infection, i.e. an infectious substance. For example, the sensors 130 detect the presence of level of a bacteria, a biofilm, an endotoxin or any other substance related to an infection.

The elongate probe 120 includes a number (e.g., 1, 2, 5, 10, or more) of light disseminating devices 140 configured to disseminate or project light 141 away from the elongate probe 120, such as laterally and/or radially away from the exterior surface 120C of the elongate probe 120. The light disseminating devices 140 are generally configured to expose substances, including infectious substances, surrounding (or generally in the proximity or presence of) the elongate probe 120 to the light 141. The light disseminating devices 140 are operatively coupled with the console 115 so that the console 115 may activate and/or deactivate the light disseminating devices 140. In some embodiments, the light disseminating devices 140 may be light sources, e.g., light emitting diodes (LEDs) electrically coupled with the console 115. In other embodiments, the light disseminating devices 140 may be optically coupled to a light source, e.g., the console 115 may include a light source and the light disseminating devices 140 may be optically coupled with the console 115.

The light 141 is generally configured to denature, disrupt, kill, remove (or otherwise reduce harmful effects of) substances exposed to the light 141. In some embodiments, the light 141 may be composed of (or include) wavelengths within at least one of a blue, violet, or ultraviolet spectrum. In some embodiments, the light 141 may includes wavelengths between about 100 nm and 280 nm.

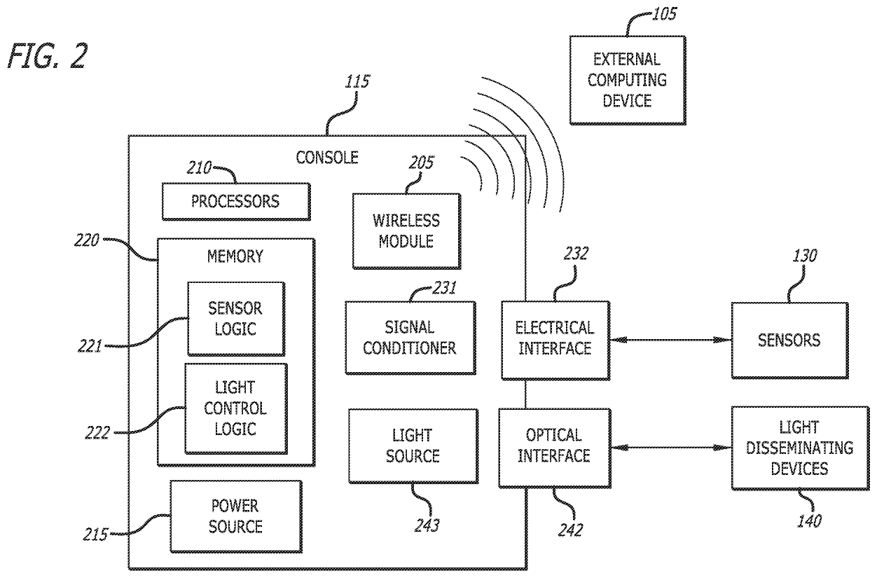

FIG. 2 illustrates a block diagram of the console 115, according to some embodiments. The console 115 is generally configured to govern the operation of the system 100. The console 115 includes a processor 210 and memory 220 (e.g., a non-transitory computer-readable medium) having logic stored thereon. In the illustrated embodiment, the logic includes sensor logic 221 and light control logic 222. The console 115 is powered via a power source 215 (e.g., a battery). The console 115 includes a wireless module 205 to facilitate wireless communication with the external computing device 105.

In some embodiments, the console 115 may include a user interface 202 configured to directly receive input from a clinician and provide output to the clinician. For example, the user interface 202 may include a number of buttons (not shown), such as an on/off button and/or light activation/deactivation button. Similarly, the user interface 202 may include a number of illuminating devices, e.g., lights or LEDs, (not shown) to indicate a status of the console 115. For example, a first illuminating device may be illuminated when the system 100 is in operation. Similarly, a second illuminating device may be illuminated when the light disseminating devices 140 are activated.

The console 115 includes an electrical interface 232 (e.g., an electrical connector) to define operative coupling between the sensors 130 and the console 115. A signal conditioner 231 converts electrical signals from the sensors 130 to digital data for processing by the processor 210 according to the sensor logic 221. Similarly, in some embodiments, the console 115 includes an optical interface 242 (e.g., a fiber optical connector) to define optical coupling between the light disseminating devices 140 and the console 115. A light source 243 may provide the light 141 to the light disseminating devices 140 according to the light control logic 222.

The sensor logic 221 is generally configured to monitor the status (e.g., infection status) of the patient and/or a treatment received by the patient via signals received from the sensors 130. The sensor logic 221 receives sensor data from the signal conditioner 231 and processes the sensor data to determine if activation of the light disseminating devices 140 may provide a benefit to the patient.

By way of one example, the sensor logic 221 may process the sensor data to determine a magnitude of a physiological parameter of the patient, e.g., a temperature of the patient. The sensor logic 221 may compare the magnitude of the physiological parameter with a limit of the physiological parameter stored in the memory 210. In some instances, the sensor logic may determine that the magnitude exceeds the limit (e.g., the patient has an abnormally high temperature as may be caused by an infection). In such an instance, the sensor logic 221 may determine that activating the light disseminating devices 140 may reduce the infection (or the effects of the infection).

By way of another example, the sensor logic 221 may detect the presence or level of the substance adjacent the elongate probe where the substance is associated with an infection. The sensor logic 221 may compare the presence or level of the substance with a limit of the substance stored in the memory 210. In some instances, the sensor logic may determine that the presence or level of the substance exceeds the limit (e.g., presence or level of the substance indicates an infection). In such an instance, the sensor logic 221 may determine that activating the light disseminating devices 140 may reduce the level of the substance and thereby, reduce the infection.

The light control logic 222 is generally configured to activate and/or deactivate the light disseminating devices 140 based on a triggering event. In some embodiments, the triggering event may include physical manipulation of the system module 110 such as pressing the activation button of the user interface 202. The triggering event may include other interactions with the system 100, such as flushing a catheter, delivering a medication, or attaching a device to an extension leg hub connector, for example. In some embodiments, the triggering event may include input received from the external computing device 105. For example, the clinician may activate the light disseminating devices 140 by pressing an icon on a cell phone.

In some embodiments, the triggering event may include data received from the sensor logic 221, such as a physiological parameter or a substance level exceeding a predefined limit as discussed above. In some embodiments, the light control logic 222 may receive data from the sensor logic 221 that the physiological parameter or a substance level has been reduced below the predefined limit, and in response thereto, the light control logic 222 may deactivate the light disseminating devices 140. In some embodiments, the light control logic 222 may automatically deactivate the light disseminating devices 140 after a defined duration.

In some embodiments, the sensor logic 221 may provide output to the external computing device 105, where the output contains information pertaining the magnitude of a physiological parameter and/or the presence or level of the substance. In response to the output, the clinician may provide a triggering event to the light control logic 222. Similarly, the light control logic 222 may provide output to the external computing device 105 indicating an activated or deactivated state of the light disseminating devices 140.

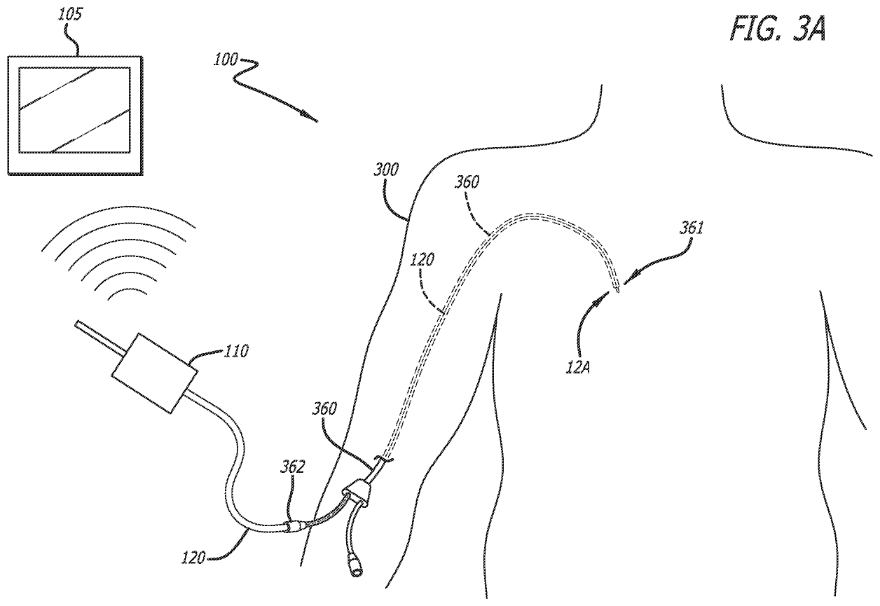

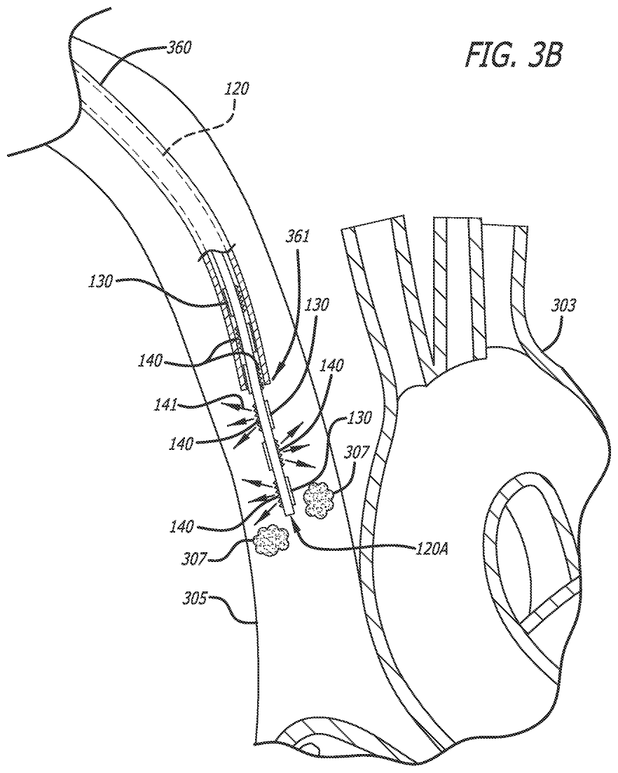

FIG. 3A illustrates the system 100 in use within a patient 300, according to some embodiments. The system 100 includes the elongate probe 120 in the form of a stylet disposed within a lumen of a catheter 360 (e.g., a peripherally inserted central catheter). The elongate probe 120 enters the lumen at a hub 362 of an extension leg and extends along the catheter 360 to the distal end 361. The system module 110 is wirelessly coupled with the external computing device 105.

FIG. 3B is a detailed illustration of the distal end 361 of the catheter 360 and the distal end 120A of the elongate probe 120 disposed within a blood vessel 305, e.g., a superior vera cava adjacent the heart 303, for example. A distal portion of the catheter 360 is shown cut away to reveal the elongate probe 120 therein. As shown is a distal portion of the elongate probe extended distally beyond the distal end 361 of the catheter 360.

The elongate probe 120 includes a number of sensors 130 disposed along the distal portion including a subset of the sensors 130 disposed within the catheter lumen. Also shown are a number of the light disseminating devices 140 disposed along the distal portion including a subset of the light disseminating devices 140 within the catheter lumen. The sensors 130 detect the presence of a substance 307, such as bacteria within the blood, for example. In response, the light disseminating devices 140 are activated to expose the substance 307 to the light 141. As such, the light 141 may denature the substance 307, e.g., damage or kill the bacteria.

Although not shown, the sensors 130 within the catheter lumen may detect a bio-film disposed on an inside surface of a luminal wall of the catheter 360. In response, the light disseminating devices 140 within the lumen may be activated to expose the bio-film to the light 141. As such, the light 141 may denature the biofilm, e.g., break up or remove the bio-film.

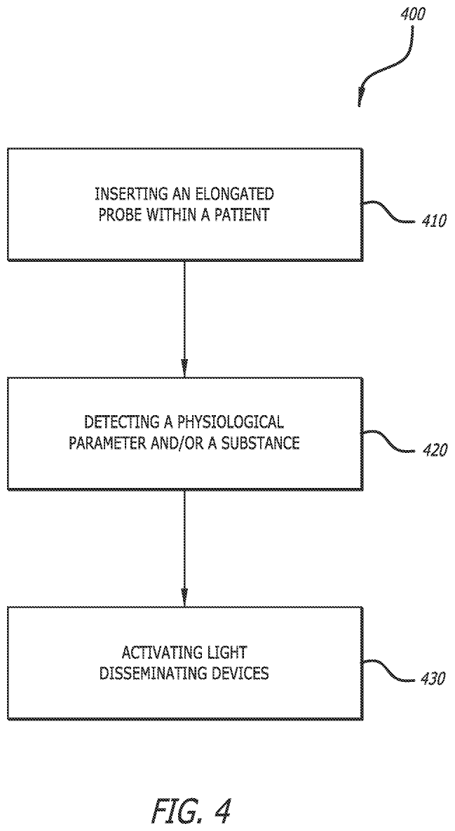

FIG. 4 illustrates a flow chart of an exemplary method 400 for treating a patient infection, according to some embodiments. The method 400 includes inserting an elongate probe within a patient (block 410), where the elongate probe has a number of the sensors. Inserting the elongate probe within the patient may include (i) inserting the elongate probe within a vasculature of the patient and/or (ii) inserting the elongate probe within a catheter lumen.

The method 400 further includes detecting, via the number of sensors, a physiological parameter and/or an infectious substance (block 420). In some embodiments, the physiological parameter may exceed a predefined limit. In some embodiments, detecting the infectious substance may include detecting the level of the infectious substance that exceeds a predefined level limit.

The method 400 includes activating a number light disseminating devices coupled with the elongate probe (block 430), where a light disseminated by the light disseminating devices is configured to reduce the physiological parameter and/or denature the infectious substance.

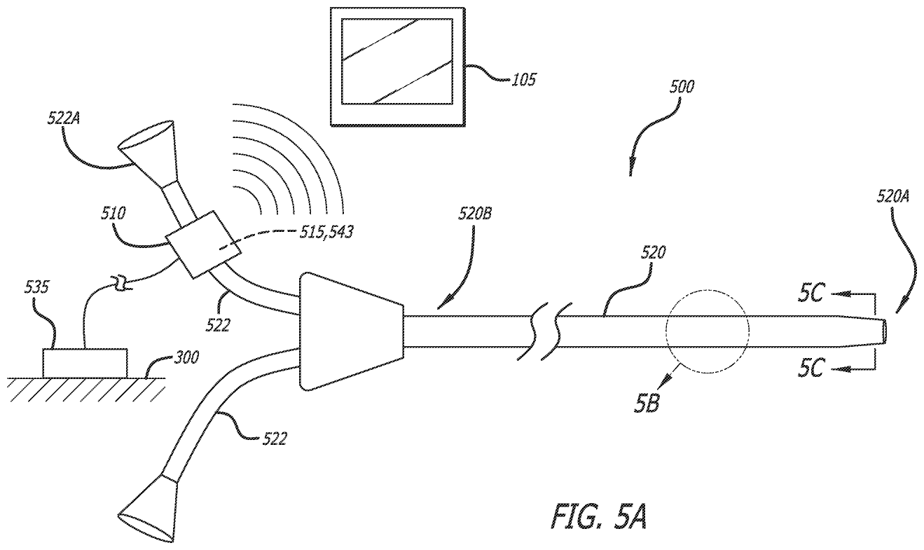

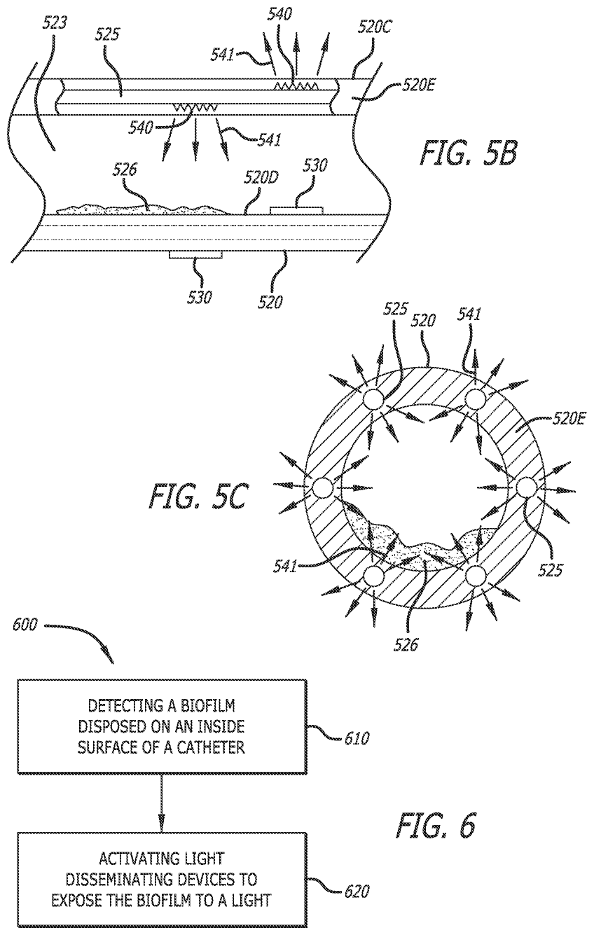

FIGS. 5A-5C illustrate another embodiment of a medical system that can, in certain respects, resemble components of the medical system described in connection with FIGS. 1-3B. It will be appreciated that all the illustrated embodiments may have analogous features. Accordingly, like features are designated with like reference numerals, with the leading digits being “5.” For instance, the system module is designated as “110” in FIGS. 1-3B, and an analogous system module is designated as “510” in FIGS. 5A-5C. Relevant disclosure set forth above regarding similarly identified features thus may not be repeated hereafter. Moreover, specific features of the medical system and related components shown in FIGS. 1-3B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the medical system of FIGS. 5A-5C. Any suitable combination of the features, and variations of the same, described with respect to the medical system and components illustrated in FIGS. 1-3B can be employed with the medical system and components of FIGS. 5A-5C, and vice versa. FIG. 5A is a plan view illustration of the system 500. FIG. 5B is a detailed cross-sectional side view illustration of a portion of the catheter 520 and FIG. 5C is a cross-section end view of the catheter 520.

Similar to the system 100, the medical system (system) 500 is generally configured to provide or enhance a treatment for a patient. More specifically, the system 500 is configured to inhibit or reduce infection or the symptoms of infection, such as sepsis, for example. The system 500 may detect the presence or level of the infection and project a light onto infection substances to denature or otherwise reduce the effects of the infection.

Referring to FIG. 5A, the system 500 includes a system module 510 coupled with a catheter 520 defining a distal end 520A and a proximal end 520B, where the catheter 520 may in some respects resemble the components, features, and functionality of the elongate probe 120. The catheter 520 is configured for insertion into a patient body where the desired effects of the light may be applied. The system module 510 is configured for use outside the patient body and may be coupled with the catheter 520 adjacent the proximal end 520B. In various embodiments, the system module 510 may be coupled a catheter tube, an extension leg, a connector hub or any suitable portion of the catheter disposed outside of the body during use.

The system module 510 includes a console 515 which in some embodiments, may be configured to exchange data and/or information with an external computing device 105, such as a network computer, a tablet, or a cell phone, for example. In some embodiments, the computing device 105 may be coupled with or otherwise associated with an electronic medical records system.

The system module 510 may be configured to selectively attach to and detach from the catheter 520. In some embodiments, the catheter 520 may be configured for single use and the system module 510 for multiple uses. For example, the system module 510 may be configured to individually couple with a number (e.g., 2, 5, 10, 20 or more) of catheters 520.

The system 500 may be a central catheter configured for placement within the patient body for an extend period of time, e.g., several days. In some embodiments, the system 500 may be configured to extend the useful life of the catheter 520. For example, the system 500 may be configured to maintain a patency of the catheter 520.

Referring to the FIG. 5B, the system 500 includes a number (e.g., 5, 2, 3, 4 or more) of sensors 530 configured to detect or measure parameters associated with the patient. The sensors 530 are communicatively coupled with the console 515 so that the console 515 may receive and process electrical signals from the sensors 530. In some embodiments, at least a subset of the sensors 530 may be disposed along the catheter 520. In some embodiments, at least a subset of the sensors 530 may be located on an exterior surface 520C of the catheter 520. The sensors 530 may take on any suitable form factor. For example, a sensor 530 may take form of a patch disposed on a side of the catheter 520 or a sensor 530 may take form of a band extending around the catheter 520. In some embodiments, at least a subset of the sensors 530 may be located on an interior luminal surface 520D of the catheter 520.

The sensors 530 are configured to operatively couple with the patient via any suitable mechanism, i.e., the sensors 530 may fluidly, optically, electrically, thermally, physically, or biologically couple with the patient. In some embodiments, a subset 535 of the sensors 530 may be physically coupled with an external skin surface of the patient.

In some embodiments, at least a subset of the sensors 530 are configured to detect or measure one or more physiological parameters of the patient. In some embodiments, the physiological parameters may include a change in color of bodily tissue, such as a redness of the skin for example. The physiological parameters may include a change in shape of bodily tissue, such as a swelling, for example. Additional physiological parameters may include a temperature, a pH, a white blood cell count, a blood pressure or a lactate level of the patient. In some embodiments, the physiological parameters may be symptomatically associated with an infection of the patient.

In some embodiments, at least a subset of the sensors 530 are configured to detect the presence or level of a substance associated with an infection. For example, the sensors 530 detect the presence of level of a bacteria, a biofilm, an endotoxin or any other substance related to an infection. In some embodiments, at least a subset of the sensors 530 are configured to detect a biofilm on an interior luminal surface of the catheter 520.

The catheter 520 includes a number (e.g., 5, 2, 5, 10, or more) of light disseminating devices 540 configured to disseminate or project light 541 away from the catheter 520, such as laterally and/or radially away from the exterior surface 520C of the catheter 520. In some embodiments, at least a subset of the light disseminating devices 540 may be configured to project light 541 radially inward from an interior luminal wall surface 520D. In general, the light disseminating devices 540 are configured to expose substances surrounding the catheter 520 and/or within the catheter lumen 523 to the light 541. The light disseminating devices 540 are operatively coupled with the console 515 so that the console 515 may activate and/or deactivate the light disseminating devices 540.

Referring to FIGS. 5B and 5C, in the illustrated embodiment, the catheter 520 includes a number (2, 3, 4, 5 or more) of optical fibers 525 extending along the length of the catheter 520. In some embodiments, the optical fibers 525 may be embedded within a luminal wall 520E. The optical fibers 525 are optically coupled with the light source 543 of the console 515 are a configured to propagate the light 541 along the catheter 520. The light disseminating devices 540 may include reflective devices disposed along the optical fibers 525, where the reflective devices are configured to direct the light 541 laterally or radially away from the optical fibers 525. For example, light disseminating devices 540 may expose a biofilm 526 to the light 541 to denature the biofilm 526 or otherwise remove or reduce the occluding effects of the biofilm 526.

The light 541 is generally configured to denature (or otherwise reduce harmful effects of) substances exposed to the light 541. In some embodiments, the light 541 may be composed of (or include) wavelengths within at least one of a blue, violet, or ultraviolet spectrum. In some embodiments, the light 541 may include wavelengths between about 100 nm and 280 nm.

FIG. 6 illustrates a flow chart of an exemplary method 600 for maintaining patency of a catheter, such as a central catheter, for example. The method 600 includes detecting, via a number of sensors coupled with an inside surface of a luminal wall the catheter, a biofilm disposed along the inside surface (block 610). The method 600 further includes activating a number light disseminating devices (block 620) coupled with the catheter to project a light away from the inside surface onto the biofilm, where the light is configured to denature the biofilm.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical system, comprising:
an elongate probe configured for insertion into a patient body, the elongate probe including a number of light disseminating devices;
a number of sensors operatively coupled with the patient body; and
a system module coupled with the elongate probe, the system module including a console operatively coupled with the sensors and the light disseminating devices, the console including a processor and a memory having stored thereon logic that, when executed by the processor, causes operations of the system that include:
receiving a sensor signal from the sensors; and
activating the light disseminating devices in response to a triggering event,
wherein a light disseminated by the light disseminating devices is configured to denature an infectious substance adjacent the elongate probe.

2. The system according to claim 1, wherein light is composed of wavelengths within at least one of a blue, violet, or ultraviolet spectrum.

3. The system according to claim 1, wherein the triggering event includes a physical manipulation of the system module by a clinician.

4. The system according to claim 1, wherein:
the console is communicatively coupled with an external computing device, and
the operations further include:
receiving input from the external computing device; and
transmitting system operational information to the external computing device.

5. The system according to claim 4, wherein the triggering event includes an input received from the external computing device.

6. The system according to claim 1, wherein:
the system module is configured to selectively attach to and detach from the elongate probe, and
attachment of the system module to the elongate probe includes operatively coupling the console with the sensors and the light disseminating devices.

7. The system according to claim 1, wherein at least a subset of the sensors are coupled with an outside surface of the elongate probe.

8. The system according to claim 1, wherein the light disseminating devices are configured to project a light radially away from the elongate probe.

9. The system according to claim 1, wherein the elongate probe includes a stylet configured for placement within a lumen of a catheter.

10. The system according to claim 1, wherein:
the elongate probe includes a number of optical fibers extending along the elongate probe, the optical fibers including the light disseminating devices;
the console includes a light source coupled with the optical fibers; and
activating the light disseminating devices includes activating the light source.

11. The system according to claim 10, wherein:
the elongate probe includes a catheter, and
the optical fibers are embedded within a luminal wall of the catheter.

12. The system according to claim 11, wherein at least a subset of the sensors are coupled to an inside luminal wall surface of the catheter.

13. The system according to claim 12, wherein the light disseminating devices are configured to project the light radially inward from the luminal wall.

14. The system according to claim 13, wherein activating the light disseminating devices is configured to denature a biofilm disposed within the lumen.

15. The system according to claim 1, wherein:
the operations further include comparing a magnitude of a physiological parameter of the patient based on the sensor signal with a limit of the physiological parameter stored in the memory, and
the triggering event includes a result of the comparison indicating that the magnitude exceeds the limit.

16. The system according to claim 1, wherein at least a subset of the sensors are coupled to a skin surface of the patient.

17. The system according to claim 16, wherein the physiological parameter includes at least one of a redness skin color, a swelling, a temperature, a pH, a white blood cell count, a blood pressure or a lactate level.

18. The system according to claim 1, wherein:
the sensor signal includes a detection level of the infectious substance adjacent the elongate probe,
the operations further include comparing the detection level with a detection level limit stored in the memory, and
the triggering event includes a result of the comparison indicating that the detection level exceeds the detection level limit.

19. A method of treating a patient infection, comprising:
inserting an elongate probe within a vasculature of the patient, the elongate probe including a number of sensors;
detecting via the number of sensors at least one of (i) a physiological parameter of the patient exceeding a predefined limit or (ii) a detection level of an infectious substance exceeding a predefined detection level limit; and
activating a number light disseminating devices coupled with the elongate probe,
wherein the light disseminating devices disseminate a light configured to denature the infectious substance.

20. A method of maintaining patency of a catheter, comprising:
detecting, via a number of sensors coupled with an inside surface of a luminal wall the catheter, a biofilm disposed along the inside surface; and
activating a number light disseminating devices coupled with the catheter to project a light away from the inside surface onto the biofilm, the light configured to denature the biofilm.
